(19) United States
(12) Patent Application Publication
(10) Pub. No.: US 2024/0170126 A1
(43) Pub. Date: May 23, 2024

(54) Automatically Adjustable Patient Temperature Alarm Tracking Current Patient Temperature During Temperature Management Therapy

(71) Applicant: ZOLL Circulation, Inc., San Jose, CA (US)

(72) Inventor: Mark G. Mitchell, San Jose, CA (US)

(73) Assignee: ZOLL Circulation, Inc., San Jose, CA (US)

(21) Appl. No.: 18/518,371

(22) Filed: Nov. 22, 2023

Related U.S. Application Data
(60) Provisional application No. 63/427,693, filed on Nov. 23, 2022.

(51) Int. Cl. G16H 20/30; G16H 40/67

(52) U.S. Cl. CPC G16H 20/30; G16H 40/67

(57) ABSTRACT

In an illustrative embodiment, systems and methods for monitoring patient temperature during delivery of temperature management therapy include activating a patient temperature tracking mode during which a temperature threshold alert corresponding to a temperature tracking threshold is enabled, initializing the temperature tracking threshold within a predetermined value of an initial patient temperature, and repeatedly, during temperature management therapy configured to adjust the initial patient temperature to a target patient temperature, obtaining a current patient temperature based on subsequent temperature data from at least one temperature sensor and, based at least in part on the current patient temperature being closer to the target patient temperature, updating the temperature tracking threshold to be within the predetermined value of the current patient temperature.

### RELATED APPLICATIONS

The present application claims priority to U.S. Provisional Application No. 63/427,693 entitled “Automatically Adjustable Patient Temperature Alarm Tracking Current Patient Temperature During Temperature Management Therapy” and filed Nov. 23, 2022. All above identified applications are hereby incorporated by reference in their entireties.

### BACKGROUND

In various clinical situations, it is desirable to warm, cool or otherwise control the body temperature of a subject. For example, hypothermia can be induced in humans and some animals for the purpose of protecting various organs and tissues (e.g., heart, brain, kidneys) against the effects of ischemic, anoxic, or toxic insult. For example, animal studies and/or clinical trials suggest that mild hypothermia can have neuroprotective and/or cardioprotective effects in animals or humans who suffer from ischemic cardiac events (e.g., myocardial infarction, acute coronary syndromes, etc.), postanoxic coma after cardiopulmonary resuscitation, traumatic brain injury, stroke, subarachnoid hemorrhage, fever and neurological injury.

One method for inducing hypothermia is by intravascular or endovascular temperature management during which a heat exchange catheter is inserted into a blood vessel and a thermal exchange fluid is circulated through a heat exchanger positioned on the portion of the catheter that is inserted in the blood vessel. As the thermal exchange fluid circulates through the catheter's heat exchanger, it exchanges heat with blood flowing past the heat exchanger in the blood vessel. Such a technique can be used to cool the subject's flowing blood thereby resulting in a lowering of the subject's core body temperature to some desired target temperature. Endovascular temperature management is also capable of warming the body and/or of controlling body temperature to maintain a monitored body temperature at some selected temperature. If a controlled rate of re-warming or re-cooling from the selected target temperature is desired, that too can be accomplished by carefully controlling the amount of heat added or removed from the body and thereby controlling the temperature change of the patient.

### SUMMARY OF ILLUSTRATIVE EMBODIMENTS

In one aspect, the present disclosure relates to a patient temperature management system for monitoring patient temperature during delivery of temperature management therapy, the system including a non-volatile computer-readable memory storing a target patient temperature, and processing circuitry configured to perform operations including activating a patient temperature tracking mode, where, while in the patient temperature tracking mode, a temperature threshold alert corresponding to a temperature tracking threshold is enabled, initializing the temperature tracking threshold based on an initial patient temperature measured using temperature data from at least one temperature sensor, where the temperature tracking threshold is set to be within a predetermined value of the initial patient temperature, and repeatedly, during temperature management therapy configured to adjust the initial patient temperature to a target patient temperature, obtaining the current patient temperature based on temperature data from the at least one temperature sensor, and, based at least in part on determining the current patient temperature is indicative of movement toward the target patient temperature, updating the temperature tracking threshold to be within the predetermined value of the current patient temperature.

In some embodiments, the system includes a temperature management device configured to perform the temperature management therapy. The system may include at least one output device for generating an alarm for an operator of the patient temperature management system corresponding to the temperature tracking threshold. The operations may include, based at least in part on the current patient temperature being further away from the target patient temperature than a current value of the temperature tracking threshold, issuing an audible alert via the at least one output device. The system may include the at least one temperature sensor.

In some embodiments, the system includes a display device, where the patient temperature tracking mode is activated based on a user input setting at an interactive user interface presented on the display device. The operations may include presenting, at the display device, a current value of the temperature tracking threshold. Presenting the current value of the temperature tracking threshold may include presenting the current value on a line graph presenting historic values of the temperature tracking threshold. The operations may include, based at least in part on the current patient temperature being further away from the target patient temperature than a current value of the temperature tracking threshold, presenting an alert on the display device.

In some embodiments, activating the patient temperature tracking mode includes automatically activating the patient temperature tracking mode based at least in part on the initial patient temperature being at least a threshold value further away from the target patient temperature than a patient temperature limit. Activating the patient temperature tracking mode may include receiving user selection of a setting corresponding to the patient temperature tracking mode. Obtaining the current patient temperature may include continuously monitoring the current patient temperature. While in the patient temperature tracking mode, a temperature limit alert corresponding to a patient temperature limit may be disabled. When the current patient temperature meets or moves beyond the patient temperature limit, the temperature limit alert may be automatically enabled and the temperature tracking threshold may be automatically disabled. The temperature limit alert may be automatically enabled when the current patient temperature moves beyond the patient temperature limit by at least the predetermined value.

In one aspect, the present disclosure relates to a method for monitoring patient temperature during delivery of temperature management therapy, the method including activating, by processing circuitry, a patient temperature tracking mode, where, while in the patient temperature tracking mode, a temperature threshold alert corresponding to a temperature tracking threshold is enabled, initializing, by the processing circuitry, the temperature tracking threshold based on an initial patient temperature measured using temperature data from at least one temperature sensor, where the temperature tracking threshold is set to be within a predetermined value of the initial patient temperature, and repeatedly, during temperature management therapy configured to adjust the initial patient temperature to a target patient temperature, obtaining, by the processing circuitry, a current patient temperature based on temperature data from the at least one temperature sensor, and analyzing, by the processing circuitry, the current patient temperature at least in view of the target patient temperature, where the analyzing includes, based at least in part on determining the current patient temperature is indicative of movement toward the target patient temperature, updating the temperature tracking threshold to be within the predetermined value of the current patient temperature.

In some embodiments, analyzing the current patient temperature at least in view of the target patient temperature includes, based at least in part on the current patient temperature meeting or moving beyond a patient temperature limit, automatically enabling the temperature limit alert, and automatically disabling the temperature tracking threshold. A temperature management therapy apparatus may include the processing circuitry. The processing circuitry may execute a set of software instructions configured to perform at least a portion of the method. At least a portion of the processing circuitry may include programmable hardware configured to perform at least a portion of the method.

In some embodiments, while in the patient temperature tracking mode, a temperature limit alert corresponding to a patient temperature limit is disabled. The analyzing may include, based at least in part on the current patient temperature being further away from the target patient temperature than a current value of the temperature tracking threshold, issuing an alert for a caregiver or operator of the temperature management therapy.

In one aspect, the present disclosure relates to a patient temperature management system for providing temperature management therapy to a patient, the system including a temperature management device configured to control temperature of a patient, at least one temperature sensor configured to generate temperature data indicative of a patient temperature, and a non-volatile computer-readable memory storing at least one threshold patient temperature, and a set of alarm rules for generating alarms during temperature management therapy, where a first alarm rule of the set of alarm rules includes a first threshold patient temperature of the at least one threshold patient temperature. The patient temperature management system may include at least one output device for generating an alarm for an operator of the patient temperature management system, and processing circuitry configured to perform operations including receiving an initiation command, submitted by the operator of the temperature management system, for delivering a temperature management therapy to a patient, and delivering the temperature management therapy to the patient, where the delivering includes determining, using the temperature data from the at least one temperature sensor, an initial patient temperature, identifying that the initial patient temperature triggers the first alarm rule, overriding the first alarm rule with a correlating alarm rule using an override threshold temperature based on the initial patient temperature, and, over a period of time, controlling a temperature of the temperature management device to adjust or maintain the temperature of the patient in accordance with one or more parameters of the temperature management therapy, where the one or more parameters includes a target patient temperature. The operations may include periodically, during the period of time, as the first alarm rule remains overridden with the correlating alarm rule, determining, using subsequent temperature data from the at least one temperature sensor, a current patient temperature, identifying the current patient temperature is closer to the target patient temperature, and adjusting the correlating alarm rule, where the adjusting includes i) updating the override threshold temperature based on the current patient temperature, or ii) reverting from the correlating alarm rule to the first alarm rule when the current patient temperature meets or moves beyond the first threshold patient temperature.

In some embodiments, receiving the initiation command includes receiving at least a portion of the one or more parameters. The one or more parameters may include at least one of a target duration of therapy or a target rate of temperature change.

In some embodiments, the system includes at least one therapy temperature sensor configured to generate temperature data indicative of a therapy temperature. The temperature management therapy may include a hypothermia therapy, and the first threshold patient temperature may be a maximum patient temperature. The temperature management therapy may include a hyperthermia therapy, and the first threshold patient temperature may be a minimum patient temperature.

In some embodiments, the temperature management device includes an endovascular catheter. The temperature management device may include a surface heat exchange device. The temperature management device may include the processing circuitry. The operations may include receiving the temperature data via a network from a separate computing device.

In some embodiments, the system includes a temperature probe including the one or more sensors, where the temperature management device receives the temperature data from the temperature probe. The at least one temperature sensor may measure at least one of esophageal, bladder, or rectal temperature. The initial patient temperature may represent a blood temperature or a surface temperature upon the skin of the patient. The operations may include preparing, for presentation at a display of the patient temperature management system, at least one graph of patient temperature versus time.

In some embodiments, the first threshold patient temperature is user-customizable within a set range of temperatures. The first threshold patient temperature may be a minimum patient temperature, and the set range may be between 28° C. and 45° C. The override threshold temperature may be within 2° C. of the initial patient temperature. Updating the override threshold temperature based on the current patient temperature may include setting the override threshold temperature to 1° C. cooler than the current patient temperature. The override threshold temperature may be in a range between 13° C. and 31° C.

In some embodiments, reverting from the correlating alarm rule to the first alarm rule occurs when the current patient temperature moves beyond the first threshold patient temperature by a temperature buffer of at least 0.3° C. The temperature buffer may be user-configurable. Reverting from the correlating alarm rule to the first alarm rule may occur when the current patient temperature meets or moves beyond the first threshold patient temperature for at least a threshold period of time.

In some embodiments, identifying the current patient temperature is closer to the target patient temperature includes identifying the current patient temperature is at least a threshold value closer to the target patient temperature. Updating the override threshold temperature based on the current patient temperature may occur when the current patient temperature remains above the override threshold temperature for at least a threshold period of time.

In some embodiments, the first alarm rule corresponds to an audible alarm. The first alarm rule may correspond to a visual alarm. The visual alarm may be presented on a display of the patient temperature management system.

In some embodiments, overriding the first alarm rule includes prompting, at a display of the patient temperature management system, for manual override of the first alarm rule, and receiving, responsive to the prompting, a confirmation to override. Reverting from the correlating alarm rule to the first alarm rule may include receiving, via a user interface, manual instruction to revert from the correlating alarm rule to the first alarm rule. The operations may further include, while the first alarm rule remains overridden with the correlating alarm rule, presenting a visual identification of the override threshold temperature representing a temperature corresponding to the correlating alarm rule. Presenting the visual identification may include presenting the override threshold temperature on a display of the patient temperature management system. Presenting the visual identification may include labeling the override threshold temperature on the display with a different label than a static threshold temperature label presented when the first alarm rule is active. The first alarm rule may be a default alarm rule of the patient temperature management system.

In one aspect, the present disclosure relates to a system for monitoring one or more physiological parameters of a patient during delivery of a medical treatment or therapy, the system including a non-volatile computer-readable memory storing at least one target physiological parameter value, and processing circuitry configured to perform operations including activating a physiological parameter tracking mode, where, while in the physiological parameter tracking mode, a threshold alert corresponding to a physiological parameter tracking threshold is enabled, and initializing the physiological parameter tracking threshold based on an initial physiological parameter value measured using physiological data from at least one sensor, where the physiological parameter threshold is set to be within a predetermined value of the initial physiological parameter value. The medical treatment may be a temperature management therapy. The operations may include repeatedly, during the delivery of the medical treatment or the therapy, obtaining a current patient physiological parameter value based on physiological data from the at least one sensor, and, based at least in part on the current physiological parameter value being closer to a first target physiological parameter value of the at least one target physiological parameter value, updating the physiological parameter tracking threshold to be within the predetermined value of the current physiological parameter value.

In some embodiments, the physiological parameter is one of a heart rate, a blood pressure, or a breathing pattern. The physiological parameter may represent an aggregation of two or more physiological measurements.

The foregoing general description of the illustrative implementations and the following detailed description thereof are merely exemplary aspects of the teachings of this disclosure, and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1A and FIG. 1B illustrate graphic outputs comparing an example fixed low alarm limit functionality to an example temperature tracking low alarm limit functionality;

FIG. 1C and FIG. 1D present additional example graphic outputs illustrating functionality of a temperature tracking low alarm limit;

FIG. 2A-1, FIG. 2A-2, FIG. 2B-1, and FIG. 2B-2 illustrate flow charts of example methods for monitoring patient temperature using a tracking temperature threshold;

FIG. 2C illustrates a flow chart of an example method for managing alerts related to a tracking temperature threshold;

FIG. 3A through 3D illustrate example user interfaces for activating a tracking temperature threshold via a graphical display;

FIG. 3E and FIG. 3F illustrate example user interfaces demonstrating automatic reversion to a static temperature threshold from a tracking temperature threshold;

FIG. 4 illustrates a series of example user interfaces for manually disabling a temperature tracking alarm threshold and reverting to a static low temperature threshold;

FIG. 5 illustrates a block diagram of an example temperature management system; and

FIG. 6 is a diagram of an example temperature management system.

### DETAILED DESCRIPTION OF ILLUSTRATIVE EMBODIMENTS

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

During intravascular, endovascular, or surface-based temperature management, physiological alarms and/or technical alarms or alerts may be triggered based on data analysis. Physiological alarms or alerts related to patient temperature may occur when the patient temperature exceeds a high or low patient temperature threshold. Technical alarms or alerts may be triggered by console errors or other events, such as, in some examples, probe or catheter disconnection, saline loop overpressure, pump malfunction or open lid. The various alarms or alerts may be audible and/or visual.

Alerts and alarms can be used to ensure that patient safety and health is protected through swift response when technical and/or physiological problems are sensed. However, caregivers can become overwhelmed with the number and frequency of alarms, depending upon various thresholds, limits, and other triggers set within the system. The inventors recognized a need for an alert mechanism that helps to avoid alarm fatigue while maintaining safety standards for patient care.

The present disclosure relates to gathering data regarding one or more physiological parameters of a patient, monitoring one or more of those parameters, alerting caregivers regarding one or more of those parameters and/or displaying information during the course of treatment that may alter one or more of those parameters. For example, in certain embodiments this may include gathering temperature data, monitoring patient temperature, alerting caregivers regarding patient temperature, and/or displaying information during the course of temperature management therapy. The systems and methods described herein can be implemented using a variety of temperature management treatment medical devices designed to monitor and adjust a patient's temperature. Certain embodiments described herein are useful in avoiding alarm fatigue while maintaining safety standards for patient care. Certain embodiments described herein are useful in nuisance alarms/alerts in circumstances where a patient's initial physiological parameters are outside safety limits. For example, a patient in critical condition at the beginning of medical treatment (e.g., such as temperature management therapy) may be exhibiting temperature, heart rate, blood pressure, respiratory rate, oxygen saturation, and/or breathing patterns that under ordinary circumstances would be cause for grave concern. In particular, the physiological parameters may align with one or more alarm limits or alert thresholds of a medical device such that, while therapy is about to begin or has just entered initial stages, default settings of the medical device may cause warnings to be issued to caregivers regarding the patient's physiological state. However, when the physiological parameter(s) measured by the medical device are indicative of a condition requiring the therapy provided by the medical device, initial alarms, alerts, and/or warnings directed to caregivers (e.g., visual, audible, etc.) may prove more of a distraction than an actual benefit to the patient's care.

In some implementations, systems and methods are disclosed herein for providing a tracking alarm limit that is initialized to a value based on one or more patient physiological parameters measured at the beginning of medical treatment. The tracking alarm limit then updates during treatment in coordination with improvements in the physiological parameter(s) as a current value of the physiological parameter(s) moves toward a default limit associated with the physiological parameter(s). In an illustrative example, when a patient's temperature initially registers below a default low temperature limit, a tracking alarm limit may be set based on the initial patient temperature such that any worsening of patient temperature triggers an alarm or alert, but steady progress of patient temperature during warming treatment results in no alarms or alerts, despite the patient temperature potentially remaining below a default low temperature limit for a significant period of time (e.g., at least ten minutes, from ten to thirty minutes, up to an hour, or up to about 2 hours).

Generally, systems described herein may also include one or more physiological alarms or alerts including a patient temperature alarm or alert. For example, a visual temperature alert may appear next to the patient's temperature on a display screen responsive to patient temperature exceeding a high temperature limit or a low temperature limit. In another example, an audible alarm may be triggered based on the patient temperature exceeding a set limit. In certain embodiments, an alarm or alert may also be transmitted to a remote device or computing system. The alarm or alert may be transmitted wirelessly, e.g., via Wi-Fi, Bluetooth Zigbee, or other wireless connection. In certain embodiments, alarm or alert messages are transmitted via USB, Ethernet, or other wired connection.

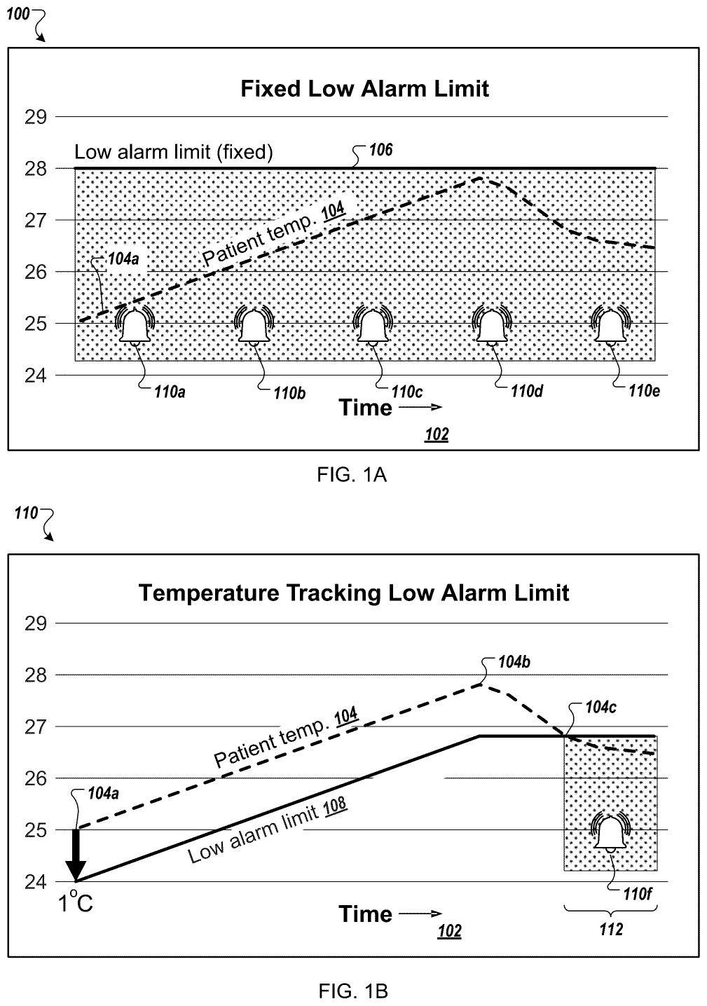

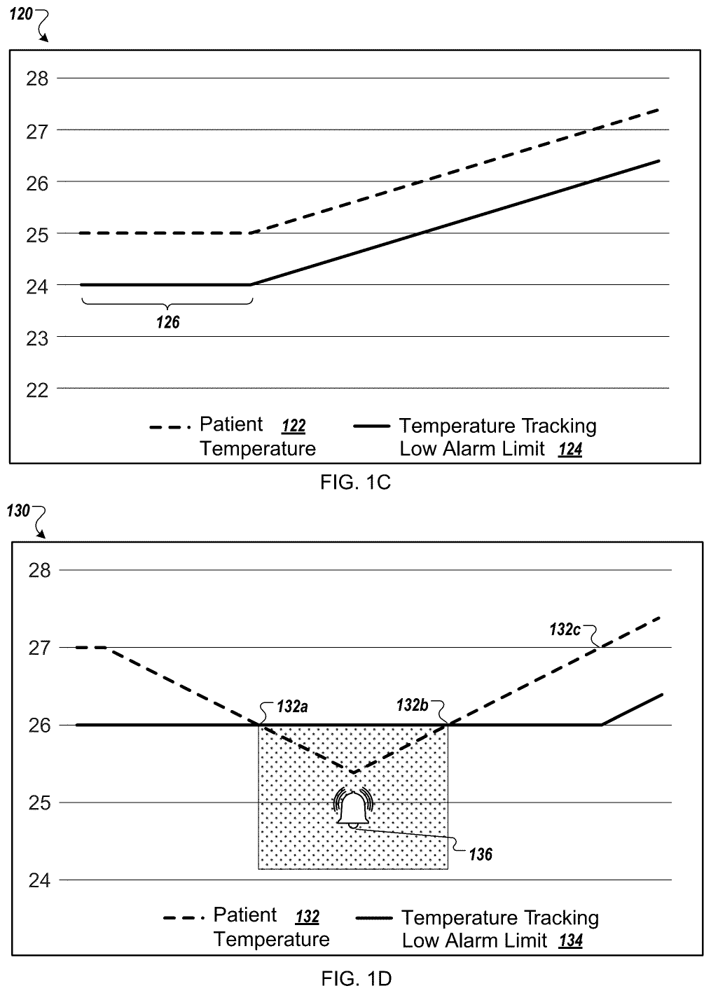

FIG. 1A and FIG. 1B illustrate side-by-side graphs 100, 110 providing comparison between the functionality of a fixed low alarm limit and an example temperature tracking low alarm limit used during temperature management therapy. As illustrated in the graphs 100, 110, a same patient temperature trajectory 104 is graphed over a timespan 102. The patient temperature 104 may be determined through one or more temperature sensors on or in the patient. The patient temperature 104 may represent a blood temperature or a surface temperature of the patient (e.g., obtained from the skin of the patient). The temperature measurements may be obtained from a variety of temperature sensors and/or medical devices, such as one or more temperature probes or sensors of a temperature management control unit and/or temperature management delivery device (e.g., an internal device such as an endovascular catheter, a surface device such as a pad, a garment, or a blanket, etc.). The at least one temperature sensor may be configured to measure esophageal temperature, bladder temperature, or rectal temperature, in some examples. The one or more temperature sensors may be located in or on the patient. In some embodiments, the temperature measurements are obtained via one or more wireless or wired connections, such as a network data connection to a remote computing system. In some embodiments, data from multiple temperature sensors are combined to determine current patient temperature. The temperature management therapy, as illustrated, involves warming a patient, e.g., induction of hyperthermia. In another example, a similar pattern may be represented for a therapy involving cooling a patient, e.g., induction of hypothermia.

Turning to FIG. 1A, the patient temperature 104 is presented in comparison to a fixed low alarm limit 106 of 28° C. As shown, an initial patient temperature 104a is three degrees below the low alarm limit 106 at 25. Thus, as therapy progresses and the patient temperature 104 slowly rises, a series of alarms 110a through 110e are issued because, during the entire time period 102 illustrated in the graph 100, the patient temperature 104 remains below the fixed low alarm limit 106 of 28° C.

Conversely, turning to FIG. 1B, the patient temperature 104 is compared to a temperature tracking alarm limit 108. The temperature tracking alarm limit 108, in some embodiments, supersedes the low alarm limit 106. For example, when the temperature tracking alarm limit 108 is active, the low alarm limit 106 may be automatically deactivated or suppressed. The temperature tracking alarm limit 108, in some implementations, automatically activates when a patient, at the beginning of therapy, registers a temperature of more than a predetermined value below the low alarm limit 106. In some implementations, a caregiver activates the temperature tracking alarm limit 108 via user settings.

In some implementations, an initial value of the temperature tracking alarm limit 108 is initialized based on an initial patient temperature. In the illustrated example, the temperature tracking alarm limit 108 is initialized to 1° C. below the initial patient temperature 104a (e.g., 24° C.). The predetermined value of 1° C. offset from the initial patient temperature 104a, for example, may be provided in part to avoid errant alarms due to a maximum variability in temperature sensitivity. For example, if precision of the temperature sensor is ±0.3° C. (e.g., a range of 0.6° C.), then using a predetermined value of 0.3° C. offset could result in a nuisance alarm based simply on the precision of temperature measurements. In some embodiments, the predetermined value is customizable by a caregiver in medical device settings. For example, the predetermined value may be customizable within a range of 0.3° C. to 2.0° C.

As patient temperature 104 rises over time, in some embodiments, the temperature tracking low alarm limit 108 automatically adjusts to maintain the predetermined offset value. As illustrated, a distance between the graph of the patient temperature 104 and the tracking low alarm limit 108 remains at a consistent width during a period where the patient temperature 104 consistently rises (e.g., up to and including patient temperature measurement 104b). Although the graph 110 illustrates a smooth and non-delayed transition of the temperature tracking low alarm limit 108 relative to patient temperature 104, in other embodiments, the temperature tracking low alarm limit 108 may be adjusted at some time after movement of the patient temperature 104. In one example, the temperature tracking low alarm limit 108 may be adjusted when the patient temperature 104 has increased by at least a threshold value, such as at least 0.25° C. In another example, the temperature tracking low alarm limit 108 may be adjusted after the patient temperature 104 has consistently remained above 1° C. higher than the temperature tracking low alarm limit 108 for at least a predetermined period of time (e.g., 1 second, 5 seconds, etc.) and/or a predetermined number of temperature measurements (e.g., at least 3 measurements, at least 5 measurements, etc.).

In some embodiments, if and when the patient temperature 104 falls during warming therapy, the temperature tracking low alarm limit 108 remains at its most recent value. As illustrated, after a patient temperature measurement 104b, the patient temperature 104 drops and the temperature tracking alarm limit 108 remains at a same value (e.g., approximately 26.75° C.). In this manner, as illustrated, when a patient temperature 104c matches the value of the temperature tracking alarm limit 108, an alarm event 110f is triggered. As illustrated, the alarm event is triggered for at least a span of time 112 during which the patient temperature 104 continues to fall below the temperature tracking low alarm limit 108. Alarm event triggering, in some examples, can include one or more visual or text indicators on a graphic display of the medical device or a computing system connected thereto, one or more audible warnings, and/or issuing one or more remote communications such as a text message to a doctor or an alert directed to the computing system at a nursing station. Visual alerts, in some examples, can include changing a color of a portion of the graph (e.g., such as the fill within the span of time 112 of graph 110), adding a warning message to a graphic display, flashing or modulating at least a portion of the graphic display, and/or increasing prominence of the patient temperature on the screen (e.g., larger, bold, and/or a red color “26.8° C.”). The type(s) of alarm(s) triggered, in some embodiments, match the type(s) of alarm(s) triggered when the fixed low alarm limit 106 is activated. In other embodiments, due to the patient physiological parameters already being outside of a zone generally considered safe to the patient, the type(s) of alarm(s) trigger include one or more higher priority/more urgent events to match the critical nature of the patient's movement in the opposite direction of progress.

FIG. 1C and FIG. 1D present additional example graphic outputs illustrating functionality of a temperature tracking low alarm limit. A graph 120 of FIG. 1C, for example, illustrates a temperature tracking limit 124 aligned with and one degree below a patient temperature 122 for a duration of a time period, from an initial patient temperature of 25° C. to an end patient temperature (or current patient temperature) of about 27.5° C. As illustrated in the graph 120, despite the patient temperature 122 failing to increase during a first time segment 126, the temperature tracking alarm limit 124 does not change, and no alarm would be issued.

Turning to FIG. 1D, after a temperature tracking low alarm limit 134 is initialized to 26° C. based on an initial patient temperature 132 of 27° C., the patient temperature 132 falls from 27° C. to about 25.5° C. At a patient temperature 132a of 26° C. corresponding to the temperature tracking low alarm limit 134, an alarm trigger 136 is initiated and remains active until the patient temperature 132 rises above a subsequent patient temperature 132b of 26° C. Further, as illustrated, in some embodiments the temperature tracking low alarm limit 134 remains at a same value (e.g., 26° C.) until the patient temperature 132 rises above the predetermined value of 1° C. above the temperature tracking alarm limit 134 (e.g., after reaching patient temperature 132c of 27° C.).

In certain embodiments, a patient may be monitored using a temperature tracking low alarm limit after having experienced accidental hypothermia, for example due to an event requiring rescue from extremely cold air temperatures or water temperatures. The patient, upon initiation of temperature management therapy, may have a body temperature significantly lower than that which would be commonplace in a medically-induced hypothermic state. To avoid errant alarm or alert conditions while the patient's temperature adjusts with the assistance of temperature management therapy, a temperature tracking low alarm limit may be set at least for an initial period of time. The period of time, for example, may involve moving between the current patient temperature and a body temperature in line with a minimum or lower range of temperature medically prescribed for induced hypothermia.

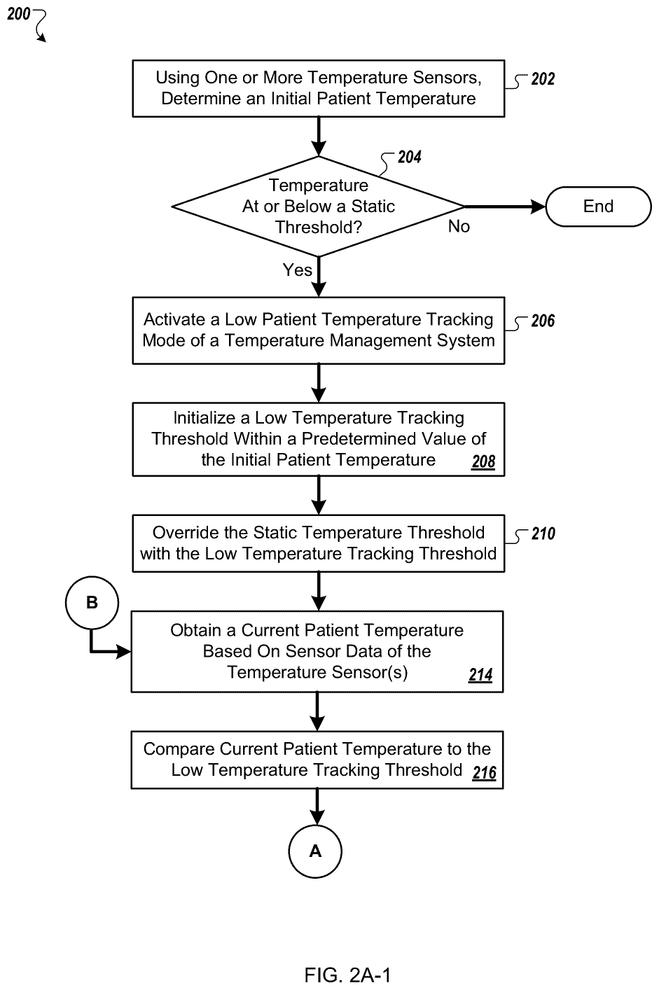

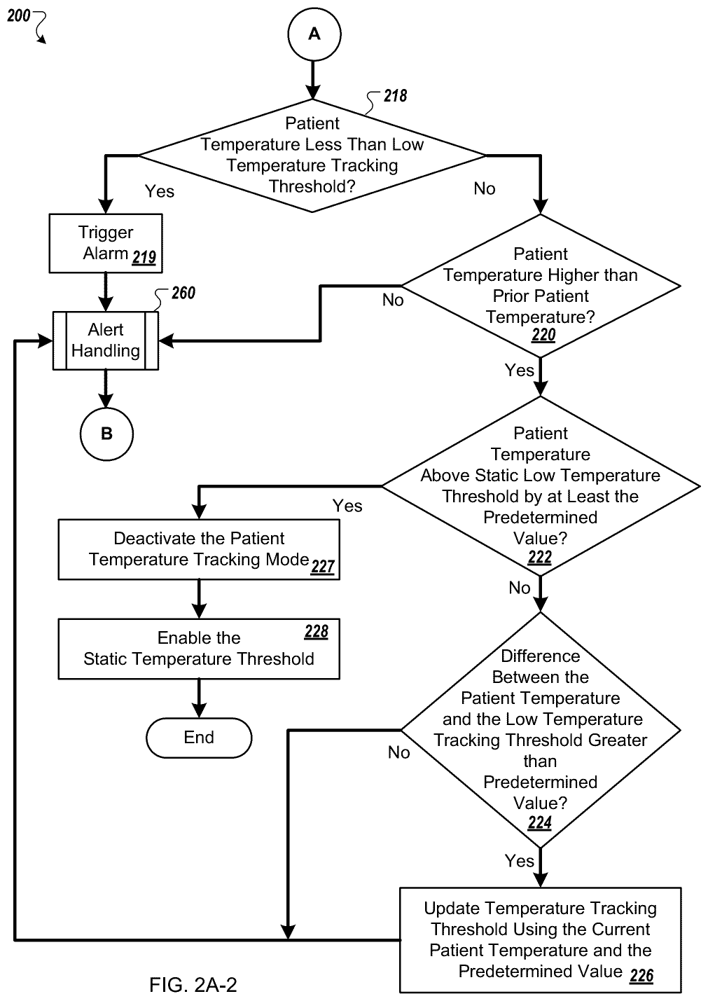

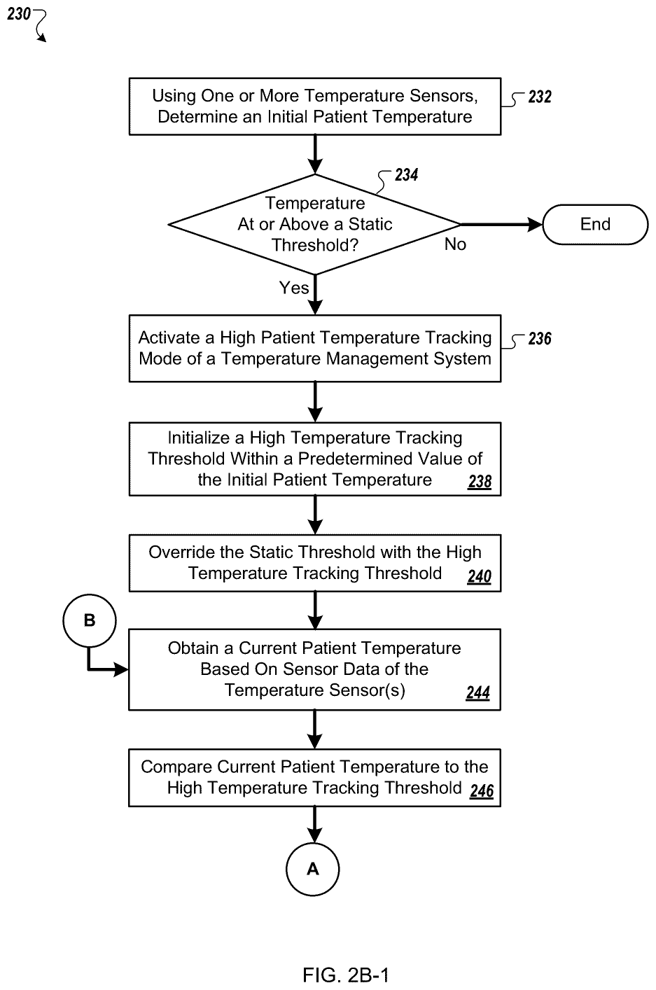

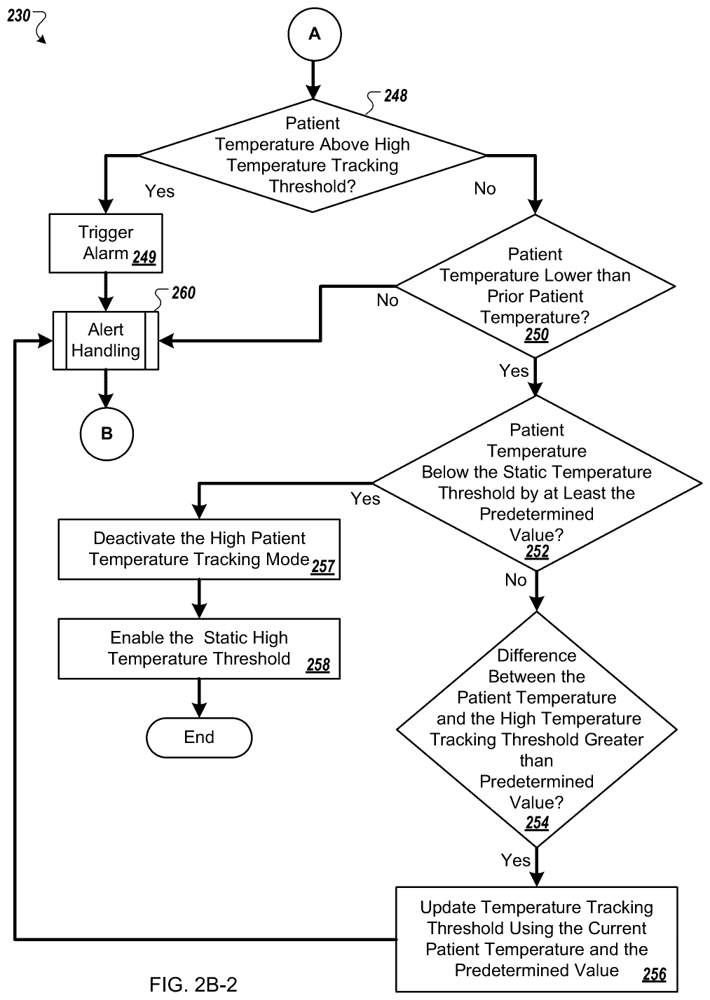

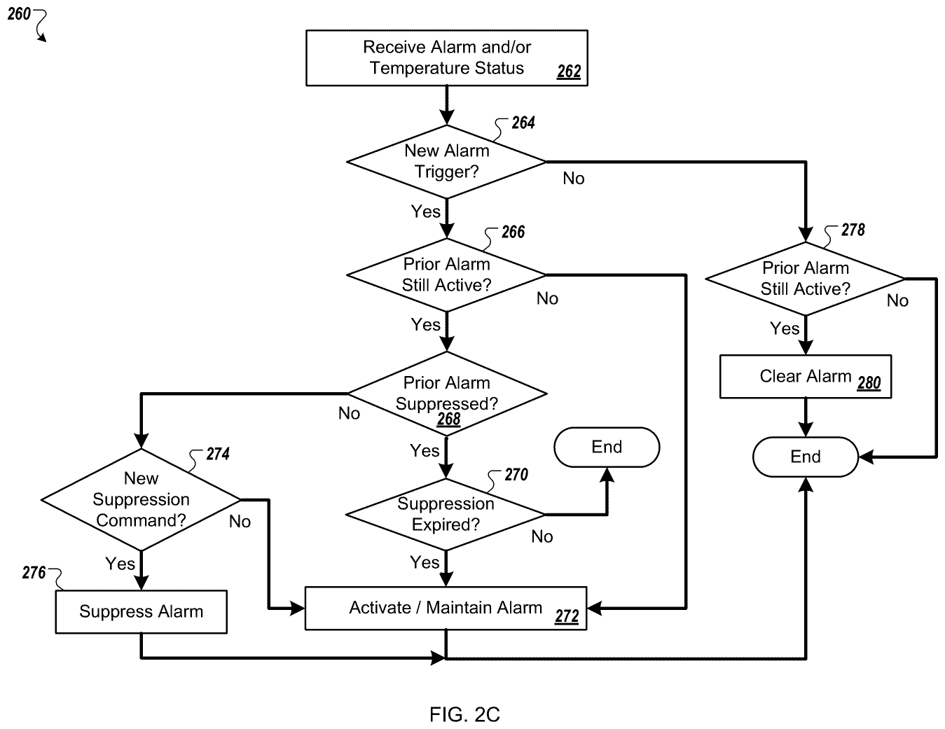

FIG. 2A-1 and FIG. 2A-2 illustrate a flow chart of an example method 200 for monitoring patient temperature using a low tracking temperature threshold. The method 200, for example, may be used to perform the functionality illustrated in the graphs of FIG. 1A through FIG. 1D.

Beginning with FIG. 2A-1, in some implementations, an initial patient temperature is determined using one or more temperature sensors (202).

As discussed above, the patient temperature may represent a blood temperature or a surface temperature of the patient. The temperature measurements may be obtained from a variety of temperature sensors and/or medical devices. The one or more temperature sensors may be located in or on the patient. Temperature measurements may be obtained via one or more wireless or wired connections. Data from multiple temperature sensors may be combined to determine the patient temperature.

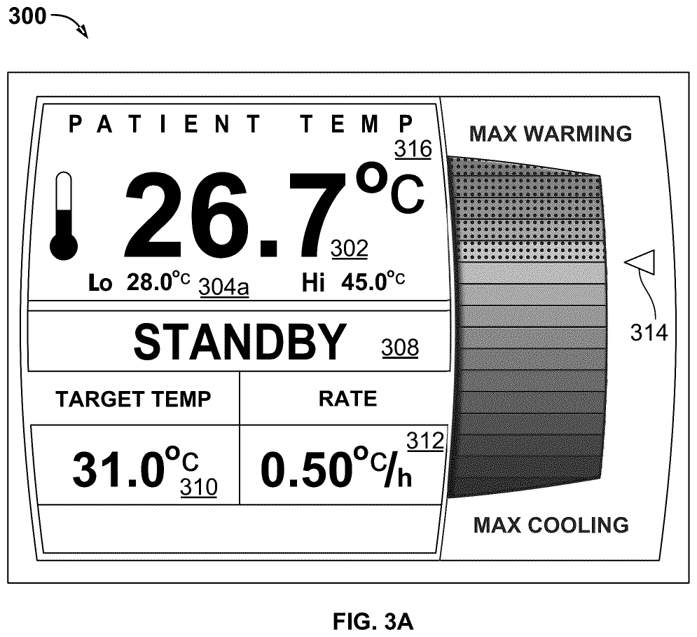

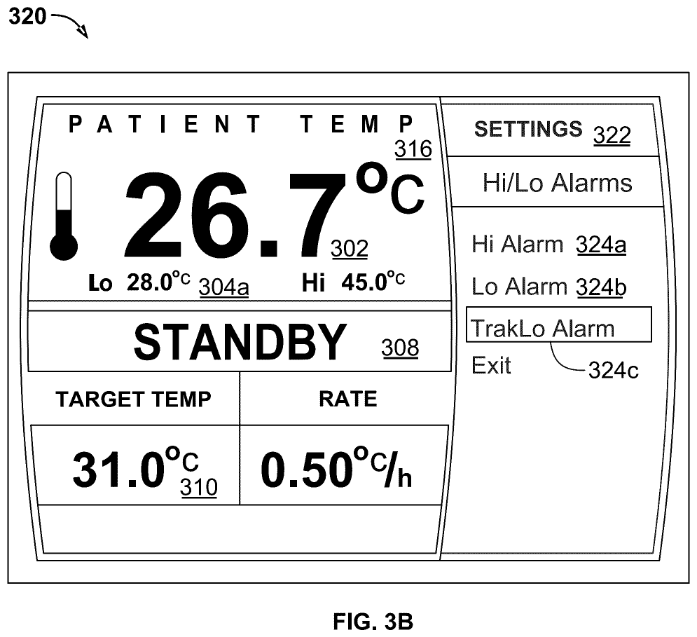

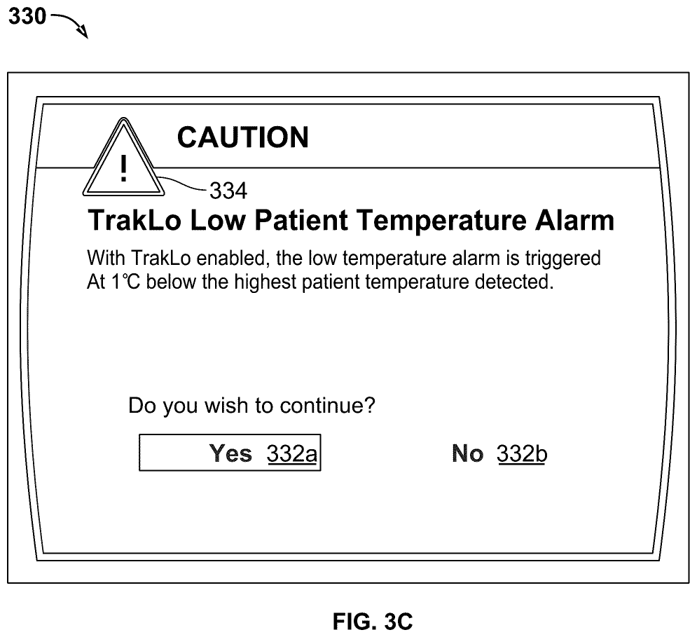

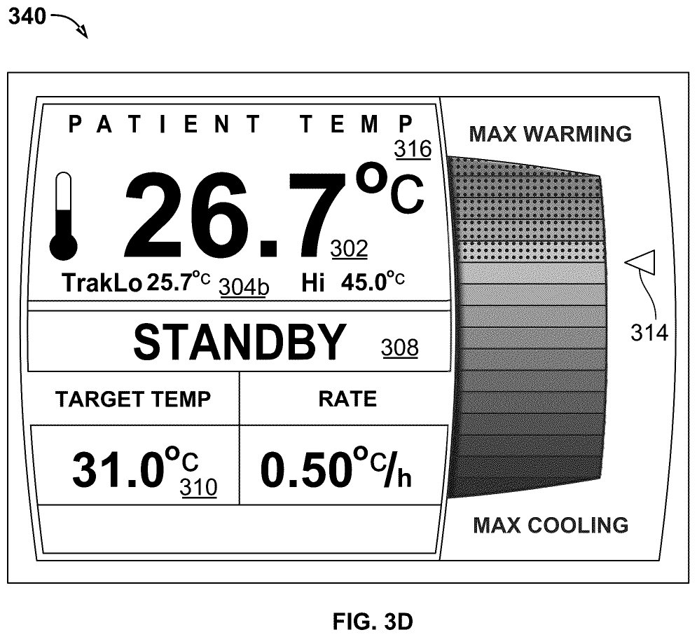

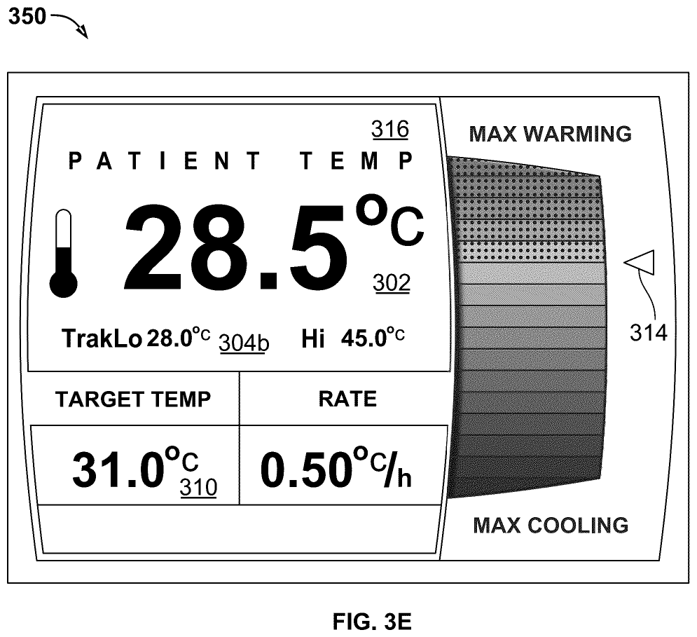

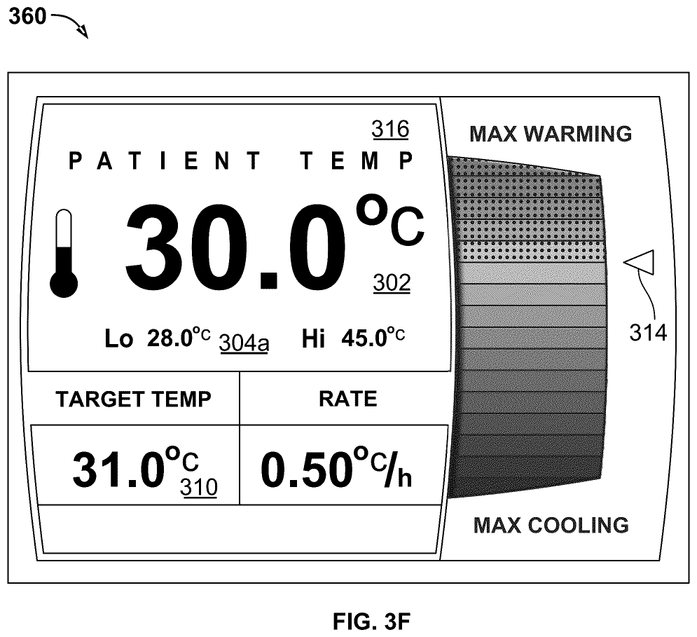

For example, as illustrated in FIG. 3A, an example display 300 indicates that a patient temperature 302 is currently 26.7° C., below a low temperature threshold of 28° C. The example display 300 further illustrates that a target temperature 310 of 31.0° C. has been set. The temperature management therapy device is in a standby mode 308, as identified in a middle of the display 300. As such, temperature management therapy is not yet active. As illustrated in a temperature level graph at the right of the display 300, a coolant bath temperature graph 314 is provided.

Returning to FIG. 2A-1, in some implementations, if the initial patient temperature is above a static low temperature threshold (204), the method 200 ends. For example, the low temperature tracking threshold may only be an option when the patient temperature is lower than a static low temperature threshold. In this manner, for example, alarms or alerts may be avoided during a period of time when the patient temperature has not yet reached the static low temperature threshold.

In some implementations, a patient temperature tracking mode of a temperature management system is activated (206). In some embodiments, the patient temperature tracking mode may be activated automatically upon recognizing that an initial patient temperature is below a default static low temperature alarm threshold. The default static low temperature alarm threshold, for example, may be set to be within a range of 28° C. to 45° C. For example, as illustrated in FIG. 3A, the display 300 indicates that a patient temperature 302 is currently 26.7° C., below a low temperature threshold of 28° C. In some embodiments, a user may manually set the patient temperature tracking mode via a settings menu upon recognizing that an initial patient temperature is below a default static low temperature alarm threshold. For example, turning to an example display 320 of FIG. 3B, a settings menu 322 has replaced the coolant bath temperature graph 314 of the display 300 of FIG. 3A. In the settings menu 322, a “TrakLo Alarm” option 324c has been selected. In some embodiments, activating the temperature tracking mode results in overriding the default static temperature limit.

Turning to FIG. 3C, in some embodiments, upon selecting the “TrakLo Alarm” option 324c, a caution display 330 is presented to the user to confirm overriding the low alarm limit. As illustrated, the display 330 presents a message stating that, by selecting the option 324c, “the low temperature alarm is triggered at 1° C. below the highest patient temperature detected.” In other examples, the message may alert the user that, by setting the “TrakLo Alarm” option 324c, the low temperature alarm is triggered at 1° C. below the highest patient temperature detected (e.g., rather than a default low threshold such as a low temperature threshold 304a of FIG. 3A). The user is prompted to confirm selection of the “TrakLo Alarm” option 324c by selecting a proceed control 332a (e.g., “Yes”) rather than a cancel control 332b (e.g., “No”).

Returning to FIG. 2A, in some implementations, a low temperature tracking threshold is initialized within a predetermined value of the initial patient temperature (208). As discussed above, the predetermined value may be set in part to avoid errant alarms or alerts due to a maximum variability in temperature sensitivity of the temperature sensor(s). The predetermined value, in some embodiments, is customizable by a user via the settings menu, such as the settings menu 322 of FIG. 3B. As illustrated in FIG. 3D, for example, the temperature tracking threshold 304b has been set to 25.7° C., 1.0° C. below the patient temperature 302.

In some implementations, a static low temperature threshold is overridden with the low temperature tracking threshold (210). For example, as seen in comparing the display 300 of FIG. 3A, to the display 240 of FIG. 3D, the low temperature threshold 304a of FIG. 3A has been replaced by the temperature tracking threshold 304b in FIG. 3D. Overriding the static temperature threshold, for example, may include suppressing, disabling, or deactivating any alarms and/or alerts associated with the static temperature threshold. For example, as discussed in relation to FIG. 1A and FIG. 1B, the alarms 110a through 110e of FIG. 1A would not be triggered due to deactivating the monitoring related to the static low alarm limit when using the temperature tracking alarm limit 108 of FIG. 1B instead.

In some implementations, a current patient temperature is obtained based on sensor data of the one or more temperature sensors (214) and the current patient temperature is compared to the low temperature tracking threshold (216). As discussed above, the current patient temperature may be a combination of multiple temperatures determined by multiple sensors. Further, in some embodiments, the current patient temperature may be determined by multiple temperature measurements obtained by the temperature sensor(s) over time. Temperature management therapy, for example, may become active by a user submitting an initiation command at a user interface. Further to the example, the initiation command may include a set of temperature management therapy parameters including at least one target temperature for adjusting a current temperature of the patient. As illustrated in FIG. 3A, for example, the target temperature of 31.0° C. has been set. Further, the temperature management therapy parameters may include a target rate of patient temperature change, (e.g., such as a rate 312 of 0.50° C./hour illustrated in FIG. 3A) and/or a target duration of treatment. In some embodiments, temperature management therapy may be initiated by powering on a temperature management therapy device (e.g., initiating a cooling or heating feature). In some examples, the temperature management device may include an endovascular catheter and/or a surface heat exchange device such as a liquid-filled pad, a garment, or a blanket.

Turning to FIG. 2A-2, if the patient temperature is less than the low temperature tracking threshold (218), in some implementations, an alarm is triggered (219). As illustrated in FIG. 1, for example, the alarm 110f is issued when the patient temperature 104 drops to a current value of the tracking alarm limit 108. The alarm, for example, may include one or more visual alarms presented at a screen of a medical device, such as a screen presenting the displays illustrated in FIG. 3A through FIG. 3F. In an illustrative example, with reference to the display 340 of FIG. 3D, the patient temperature 302 may be presented flashing, in a different color (e.g., red, as illustrated in the display 300 of FIG. 3A prior to setting of the temperature tracking alarm mode), with a different background color of a “PATIENT TEMP” section 316 of the display 340, and/or with a warning symbol presented in relation to the patient temperature 302, such as a warning symbol 334 of the display 330 of FIG. 3C. In another example, the alarm may include one or more audible alarms, such as a siren, chirp, and/or verbal message emitted from a speaker of the medical device. In another example, the alarm may include one or more messages or other alerts issued to remotely located devices, such as a text message sent to a smart phone, an SMS message sent to a tablet computer, and/or an alarm notification sent to a remote patient monitoring system.

In some implementations, an alert handling routine 260 is executed to manage the alarm triggering. Turning to FIG. 2C, in some implementations, the alert handling routine 260 begins with receiving an alarm and/or temperature status (262). The alarm status, in some examples, may be an alarm trigger, an alarm clear, and/or an alarm suppress status. The temperature status, in some examples, can include a prior temperature and/or current temperature. The temperature status may further include a static temperature threshold and/or a tracking temperature threshold. The alert handling routine 260, for example, may analyze temperature information and/or act upon alarm status information to manage alarms and/or alerts.

In some implementations, if a new alarm is triggered (264), it is determined whether a prior alarm is still active (266). As patient temperature heads in a direction opposite of a desired temperature over a period of time (e.g., target temperature, static temperature threshold, etc.) involving analysis of at least two patient temperature capture cycles, for example, a first alarm condition event may be triggered based on the first movement away from desired temperature that triggers the tracking temperature threshold, then again based on a second, subsequent movement away from the desired temperature. In illustration, upon identifying that patient temperature has fallen below a low temperature tracking temperature threshold of 26° C. to 25.8° C., a first alarm event may be triggered. At a later time, upon identifying that the patient temperature has again fallen, this time to a temperature of 25.5° C., a second alarm event may be triggered. Since alarms remain active for some time to draw attention, there is a possibility, in some embodiments, that the second alarm event is triggered while one or more alarms and/or alerts are in an active state due to the first alarm event. In this manner, the prior alarm persists (e.g., is maintained or extended in time).

In some implementations, if a prior alarm is active (266), it is determined if the prior alarm had been suppressed (268). Upon recognizing the alarm or alert, in some embodiments, a user may have the ability to suppress the alarm or alert. For example, an audible alert may be muted, or a text alert may be cleared from the screen (or made smaller/placed in a manner where it no longer obscures other information).

If a prior alarm had been suppressed (268), in some implementations, it is determined whether the suppression has expired (270). In some embodiments, one or more alarms can be temporarily muted or disabled for a period of time through manual selection of a command at the user interface. For example, a caregiver may acknowledge, at the user interface, the alarm and choose to silence or dismiss the alarm temporarily. However, the alarm, in this circumstance, remains triggered and returns to presenting visual and/or audible notifications to a user after the override period of time has passed.

For example, muting the alarm may suppress audible tones for a limited period of time such as, in some examples, a matter of 30 seconds, one minute, or two minutes. While suppression is not expired, although a new alarm triggering event has been received, in some embodiments, the method 260 ends without actively releasing a new alarm and/or alert for the attention of caregivers (e.g., audible tone, pop-up window, etc.).

Conversely, if suppression of the prior alarm has expired (270), in some implementations, the alarm is activated (272). In some examples, an audible tone may be sounded, a pop-up window may be presented in a display, the patient temperature may be altered in color (e.g., turned red), and/or the patient temperature may be visually accentuated (increased in size, flashing, highlighted, etc.) to draw attention to the current patient temperature.

Returning above, if the prior alarm was not suppressed (268), unless a new suppression command has been received (274), the alarm is maintained (272). A new suppression command, in some embodiments, may be entered by a caregiver, such as via a graphical user interface menu system, to suppress one or more alerts and/or alarms for a period of time. This may be done, for example, when caregivers actively recognize an issue and are working on resolving it. In some embodiments, the period of time is linked to a system event, such as, in some examples, activation of temperature management therapy and/or warming up of a temperature management therapy device (e.g., bath temperature reaching a target temperature), The period of time may be customizable.

In some implementations, if a new suppression command was received (274), the alarm is suppressed (276). For example, a suppression timer may be activated such that no audible and/or visual alarm or alert is presented for the attention of caregivers until the end of the suppression period of time.

Returning above, if no new alarm trigger has been received (264), then it is determined if a prior alarm is still active (278). In some embodiments, the method 260 is entered when no alarm condition is currently detected. For example, as illustrated in FIG. 2A-2, the alert handling process 260 may be executed when the patient temperature is below the static low temperature threshold and above the low temperature tracking threshold (218, 224, 226). In this manner, for example, the method 260 may clear any current alarm since there is no longer an active alarm condition.

In some implementations, if a prior alarm is still active (278), the alarm is cleared (280). Any visual alerts, for example, may revert to a prior state of the user interface, and any audible alerts may be silenced. Further, in the example where a notification was issued to remote system(s), a “clear” notification may be issued to alert the remote user to cessation of the alarm trigger. As illustrated in FIG. 1D, for example, the alarm condition ends after the patient temperature 132b has been exceeded.

Although described as a particular series of operations, in other implementations, the method 260 may differ. In some embodiments, the method 260 includes more or fewer operations. For example, a new suppression command may be received (274) without a corresponding new alarm trigger (264) based on a user interface setting, as described above. In some embodiments, portions of the method 260 are performed in a different order or concurrently. Other modifications of the method 230 are possible while remaining within the scope and spirit of the present disclosure.

Returning to FIG. 2A-2, in some implementations, after triggering the alarm (219), the method 200 returns to obtaining the current patient temperature (214) of FIG. 2A-1.

If, instead, the patient temperature is not less than the low temperature tracking threshold (218), it is determined whether the patient temperature is higher than the previously recorded patient temperature (220).

If the patient temperature is the same as the prior patient temperature (220), in some implementations, the alert handling routine is initiated (260) and/or the method 200 returns to obtaining the current patient temperature (214) of FIG. 2A-1. As illustrated in FIG. 1C, for example, during the period of time 126 where the patient temperature 122 remains the same, the temperature tracking low alarm limit 124 similarly remains unchanged.

If the patient temperature, instead, is higher than the prior patient temperature (220), in some implementations, it is determined whether the patient temperature is above the static low temperature threshold by at least the predetermined value (222). Turning to FIG. 3E, an example display 350 illustrates a condition where the patient temperature 302 (28.5° C.) is above the static temperature threshold (e.g., the low temperature threshold 304a (28° C.) of FIG. 3A).

If the patient temperature has not yet exceeded the static low temperature threshold (222) by at least the predetermined value, in some implementations, it is determined whether the difference between the patient temperature and the temperature tracking threshold is greater than the predetermined value (224). As illustrated in the example 130 of FIG. 1D, for example, while an offset between the temperature tracking low alarm limit 134 and the current patient temperature 132 is less than or equal to 1° C. (e.g., from patient temperature point 132b to patient temperature point 132c), the temperature tracking low alarm limit 134 remains at a same value. In other words, the temperature tracking threshold only tracks along with movements in patient temperature when the offset of the predetermined value can be maintained. If the difference is not greater than the predetermined value, in some implementations, the method 200 returns to obtaining the current patient temperature (214) of FIG. 2A-1.

If, instead, the difference is greater than the predetermined value, in some implementations, the temperature tracking threshold is updated using the current patient temperature and the predetermined value (226) and the method 200 proceeds to activate the alert handling routine 260 and/or return to obtaining a current patient temperature (214).

In some implementations, the method 200 continues to repeat operations 214 to 224 until the patient temperature has reached or exceeded the static temperature threshold (222). When the patient temperature is at the low static temperature threshold or above the low static temperature threshold (222), in some implementations, the patient temperature tracking mode is automatically deactivated (227) and the static temperature threshold is enabled (228). Automatically reverting to the static temperature threshold, for example, may provide the benefit of avoiding the temperature tracking threshold being the cause for errant alarm conditions in circumstances where the patient is within a physiological “safe zone” in relation to temperature. Automatically reverting may include disabling higher priority types of alarm rules associated with the temperature tracking threshold and enabling a different set (or subset) of alarm rules associated with the static temperature threshold.

In other embodiments, the temperature tracking threshold is not disabled upon the patient temperature reaching the static temperature threshold but at a temperature buffer offset from the static temperature threshold. As described above, to avoid errant alarms based on a known variance in measurements by the one or more sensors, the predetermined value (e.g., 1.0° C.) or another temperature buffer value (e.g., 0.3° C.) may be used in deciding when to revert to the static temperature threshold. The temperature buffer value, in some embodiments, is user customizable. In an illustrative example, a user may be able to set a temperature buffer value between 0.3° C. and 2.0° C.

Although described as a particular series of operations, in other implementations, the method 200 may differ. In some embodiments, the method 200 includes more or fewer operations. In one example, upon determining that the patient temperature is below a static threshold (204), one or more alarms and/or alerts may be triggered regarding the patient's temperature. In another example, along with reverting to the static temperature threshold, the user may be alerted to this update via an audible and/or visible message, such as a request for acknowledgement/approval that the temperature tracking threshold be replaced with the static temperature threshold. In another example, the low temperature tracking threshold setting may revert to the static low temperature threshold only after a predetermined period of time has elapsed during which patient temperature maintains a current value or increases in temperature. The predetermined period of time, in some examples, may include at least 1 second, at least 5 seconds, or up to 30 seconds. In a further example, activating the patient temperature tracking mode of a temperature management system may include determining whether the temperature tracking threshold is outside an acceptable range of values. In an illustrative example, a low temperature tracking threshold may be set in a range from 13° C. to 31° C. In some embodiments, portions of the method 200 are performed in a different order or concurrently. For example, the temperature tracking threshold may not be initialized (208) until the point at which temperature management therapy becomes active (212). Other modifications of the method 200 are possible while remaining within the scope and spirit of the present disclosure.

FIG. 2B-1 and FIG. 2B-2 illustrate a flow chart of an example method 230 for monitoring patient temperature using a high tracking temperature threshold. The method 230, for example, may be performed similar to the method 200 of FIG. 2A-1 and FIG. 2A-2, except for the direction of movement of temperature and the ranges of temperatures focused on.

Beginning with FIG. 2B-1, in some implementations, an initial patient temperature is determined using one or more temperature sensors (232). The initial patient temperature, for example, may be determined as described in reference to operation 202 of FIG. 2A-1.

In some implementations, if the initial patient temperature is at or below a static high temperature threshold (234), the method 230 ends. For example, the high temperature tracking threshold may only be an option when the patient temperature is higher than a static high temperature threshold.

In some implementations, a high patient temperature tracking mode of a temperature management system is activated (236). In some embodiments, the high patient temperature tracking mode may be activated automatically upon recognizing that an initial patient temperature is above a default static high temperature alarm threshold. The default static high temperature alarm threshold, for example, may be set to be within a range of about 38° C. to about 42° C. In some embodiments, activating the temperature tracking mode results in overriding the default static high temperature limit.

In some implementations, a high temperature tracking threshold is initialized within a predetermined value of the initial patient temperature (238). As discussed above, the predetermined value may be set in part to avoid errant alarms or alerts due to a maximum variability in temperature sensitivity of the temperature sensor(s). The predetermined value, in some embodiments, is customizable by a user via the settings menu.

In some implementations, a static high temperature threshold is overridden with the high temperature tracking threshold (240). Overriding the static temperature threshold, for example, may include suppressing any alarms and/or alerts associated with the static temperature threshold.

In some implementations, a current patient temperature is obtained based on sensor data of the one or more temperature sensors (244) and the current patient temperature is compared to the high temperature tracking threshold (246).

Turning to FIG. 2B-2, if the patient temperature is higher than the high temperature tracking threshold (248), in some implementations, an alarm is triggered (249). The alarm may be triggered, for example, as discussed in relation to operation 219 of FIG. 2A-2.

In some implementations, the alert handling routine 260 is executed to manage the alarm triggering.

In some implementations, after triggering the alarm (249), the method 230 returns to obtaining the current patient temperature (234) of FIG. 2B-1.

If, instead, the patient temperature is not above the high temperature tracking threshold (248), it is determined whether the patient temperature is lower than a previously recorded patient temperature (250).

If the patient temperature is the same as the prior patient temperature (250), in some implementations, the alert handling routine is initiated (260) and/or the method 230 returns to obtaining the current patient temperature (234) of FIG. 2B-1.

If the patient temperature, instead, is lower than the prior patient temperature (250), in some implementations, it is determined whether the patient temperature is at or below the static high temperature threshold (252).

If the patient temperature has not yet reached the static high temperature threshold (252), in some implementations, it is determined whether the difference between the patient temperature and the temperature tracking threshold is greater than the predetermined value (254). If the difference is not greater than the predetermined value, in some implementations, the method 230 returns to obtaining the current patient temperature (234) of FIG. 2A-1.

If, instead, the difference is greater than the predetermined value, in some implementations, the high temperature tracking threshold is updated using the current patient temperature and the predetermined value (256) and the method 230 proceeds to activating the alert handling routine 260 and/or returning to obtaining a current patient temperature (234).

In some implementations, the method 230 continues to repeat operations 234 to 244 until the patient temperature has reached or exceeded the static temperature threshold (252). When the patient temperature is at the high static temperature threshold or below the high static temperature threshold (252), in some implementations, the high patient temperature tracking mode is automatically deactivated (257) and the static high temperature threshold is enabled (258). Automatically reverting to the static high temperature threshold, for example, may provide the benefit of avoiding the temperature tracking threshold being the cause for errant alarm conditions in circumstances where the patient is within a physiological “safe zone” in relation to temperature. Automatically reverting may include disabling higher priority types of alarm rules associated with the temperature tracking threshold and enabling a different set (or subset) of alarm rules associated with the static temperature threshold.

In other embodiments, the temperature tracking threshold is not disabled upon the patient temperature reaching the static temperature threshold but at a temperature buffer offset from the static high temperature threshold. As described above, to avoid errant alarms based on a known variance in measurements by the one or more sensors, the predetermined value (e.g., 1.0° C.) or another temperature buffer value (e.g., 0.3° C.) may be used in deciding when to revert to the static temperature threshold. The temperature buffer value, in some embodiments, is user customizable. In an illustrative example, a user may be able to set a temperature buffer value between 0.3° C. and 2.0° C.

Although described as a particular series of operations, in other implementations, the method 230 may differ. In some embodiments, the method 230 includes more or fewer operations. For example, along with reverting to the static temperature threshold, the user may be alerted to this update via an audible and/or visible message, such as a request for acknowledgement/approval that the temperature tracking threshold be replaced with the static temperature threshold. In another example, the high temperature tracking threshold setting may revert to the static high temperature threshold only after a predetermined period of time has elapsed during which patient temperature maintains a current value or decreases in temperature. The predetermined period of time, in some examples, may include at least 1 second, at least 5 seconds, or up to 30 seconds. In some embodiments, portions of the method 230 are performed in a different order or concurrently. For example, the temperature tracking threshold may not be initialized (238) until the point at which temperature management therapy becomes active (232). Other modifications of the method 230 are possible while remaining within the scope and spirit of the present disclosure.

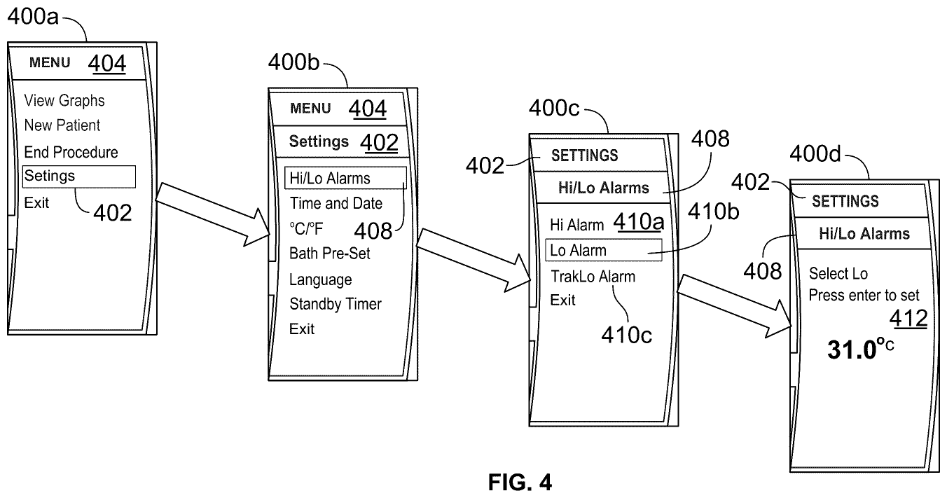

In some implementations, rather than or in addition to automatically reverting to the static temperature threshold once that temperature has been met or surpassed by the patient temperature, a user may opt to revert to the static temperature threshold manually during temperature management therapy delivery. FIG. 4 illustrates a series of example user interface graphic displays 400 for manually disabling a temperature tracking alarm threshold and reverting to a static low temperature threshold. The graphic displays 400, for example, may be presented in the settings region 322 of the graphic display 320 of FIG. 3B.

In a first graphic display 400a, a settings option 402 is selected in a main menu 404. As illustrated in a second graphic display 400b, a settings sub-menu is presented under the main menu 404, and a temperature threshold (“Hi/Lo Alarms”) option 408 is selected. The temperature threshold option 408, for example, may relate to a set of alarm rules (e.g., trigger conditions) for generating alarms during temperature management therapy. Further, each alarm rule may be associated with one or more types of alarms such as audible, visual, and/or remote messaging alarms.

In a third graphic display 400c, a temperature threshold submenu 408 presents a set of temperature threshold rules 410 including a high alarm limit 410a, a low alarm limit 410b, and a temperature tracking limit 410c. The low alarm limit 410b is selected.

In a fourth graphic display 400d, the temperature threshold submenu 408 now includes low alarm settings options 412 for the low alarm limit 410b. The user is prompted to “press enter to set” the low alarm limit (e.g., 31.0° C.). Upon entry, for example, the temperature tracking threshold may be disabled and the static low temperature threshold enabled.

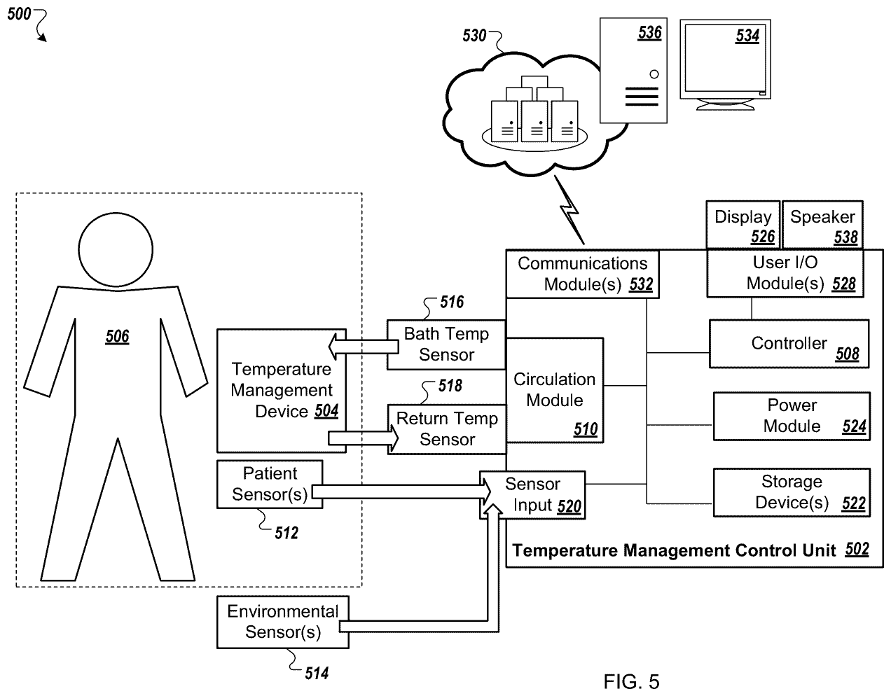

FIG. 5 is a block diagram of an example temperature management system 500 including a temperature management control unit 502 functioning in coordination with a temperature management device 504 to provide temperature management therapy to a patient 506. In use, a controller 508 of the temperature management control unit 502 controls a circulation module 510 to provide hypothermia, hyperthermia, or normothermia temperature management therapy. The controller 508 may receive feedback from a variety of sensors, such as one or more patient sensors 512 and/or environmental sensors 514. The patient sensors 512 may include one or more patient temperature probes. Further, in the circumstance that the temperature management device 504 is liquid-controlled, the circulation module 510 may include or be in communication with a liquid supply temperature sensor 516 and/or a liquid return temperature sensor 518. The controller 508 may analyze the sensor feedback (e.g., from the circulation module 510, via a sensor input module 520, and/or stored over time to storage device(s) 522), for example, to detect the onset of fever in the patient 506, leading the controller to instructing the circulation module 510 to provide additional cold, or colder, fluid, to the temperature management device 504.

In some implementations, the controller receives feedback from a power module 524 and an associated increase in the energy being drawn through the power module 524 by a cooler/heater unit of the circulation module 510.

In illustration, when the temperature management control unit 502 is being utilized for maintenance of controlled hyperthermia, the onset of fever in the patient 506 by the patient sensors 512 may be detectable as a need to decrease or cease fluid warming to the temperature management device 504 via the circulation module 510, thereby creating and an associated decrease in the energy expenditure of the power module 524.

Temperature data (e.g., related to sensor inputs 516, 518, and/or 520), in some implementations, is analyzed by the controller 508 to provide information to a user of the temperature management control unit 502 at a display 526. For example, the controller 508, via user input/output (I/O) module(s) 528, may present patient information such as a current patient temperature, a target temperature, a setting of the temperature management control unit 502 (e.g., cooling, warming, maintenance), and/or a power level may be presented as data to assist clinicians. In illustration, at least a portion of the information presented in the graph 100 of FIG. 1A, the graph 110 of FIG. 1B, the graph 120 of FIG. 1C, the graph 130 of FIG. 1D, the graphic display 300 of FIG. 3A, the graphic display 320 of FIG. 3B, the graphic display 330 of FIG. 3C, the graphic display 340 of FIG. 3D, the graphic display 350 of FIG. 3E, the graphic display 360 of FIG. 3F, and/or the graphic displays 400 of FIG. 4 may be presented at the display 526.

In some implementations, the system 500 provides alerts or alarms to the user via the user I/O modules 528. For example, visible alerts and/or alarms may be presented for caregiver attention via a display 526, while audible alerts and/or alarms may be presented via a speaker 538.

In some implementations, temperature data is provided to a remote computing device or system via one or more communication modules 532. The communication modules 532, in some examples, may be configured to establish wired or wireless communication links under a variety of communication standards. The communications may be secure and/or the information provided via the communication module(s) 532 may be encrypted. In one example, the communications module(s) 532 may be configured to communicate via a secure medical facility network. In another example, the communications module(s) 532 may be configured to communicate via a wireless data transfer interface such as a cellular data communication interface. The computing devices connecting to the communication module(s) 532 may include, in some examples, a tablet computer in wireless communication with the temperature management control unit 502, a separate medical device in wired or wireless communication with the temperature management control unit 502, or a networked server or server system (e.g., cloud computing system) such as the system 530.

Further, in some implementations, alert and/or alarm notifications may be issued to a remote computing device or system via the communications module(s) 532. The alerts and/or alarms, in some examples, may include text messages to a smart phone or paging device, short message service (SMS) messages to a computing device such as a tablet computer, and/or messages recognized by an application executing on a remote computing device such as a patient monitoring device or nursing station monitoring unit.

The system 530, in some implementations, collects temperature data and/or sensor data from the sensors 512, 514, 516, and/or 518 to provide metrics and analysis of temperature management therapy. The analysis may be performed in near-real time and/or using historical data (e.g., at the end of each stage of temperature management therapy or at the end of a temperature management therapy session). The analysis may be presented in graphs, tables, and other metrics displays, such as is found in graph 100 of FIG. 1A, the graph 110 of FIG. 1B, the graph 120 of FIG. 1C, the graph 130 of FIG. 1D, the graphic display 300 of FIG. 3A, the graphic display 320 of FIG. 3B, the graphic display 330 of FIG. 3C, the graphic display 340 of FIG. 3D, the graphic display 350 of FIG. 3E, and/or the graphic display 360 of FIG. 3F, at the display 526 of the temperature management control unit 502 and/or at a display 534 of a user computing device 536 or other remote device.

The system 530, in some implementations, enhances data displayed at the display 526 of the temperature management control unit 502. For example, the system 530 may provide additional information to the temperature management control unit 502, such as patient information, historic event data (e.g., collected prior to the patient 506 being treated using the temperature management control unit 502), and/or historic temperature data (e.g., from a different temperature management control unit 502 used to treat the patient 506).

Reference has been made to illustrations representing methods and systems according to implementations of this disclosure. Aspects thereof may be implemented by computer program instructions. These computer program instructions may be provided to a processor of a general-purpose computer, special purpose computer, or other programmable data processing apparatus and/or distributed processing systems having processing circuitry, such that the instructions, which execute via the processor of the computer or other programmable data processing apparatus, create means for implementing the functions/operations specified in the illustrations.

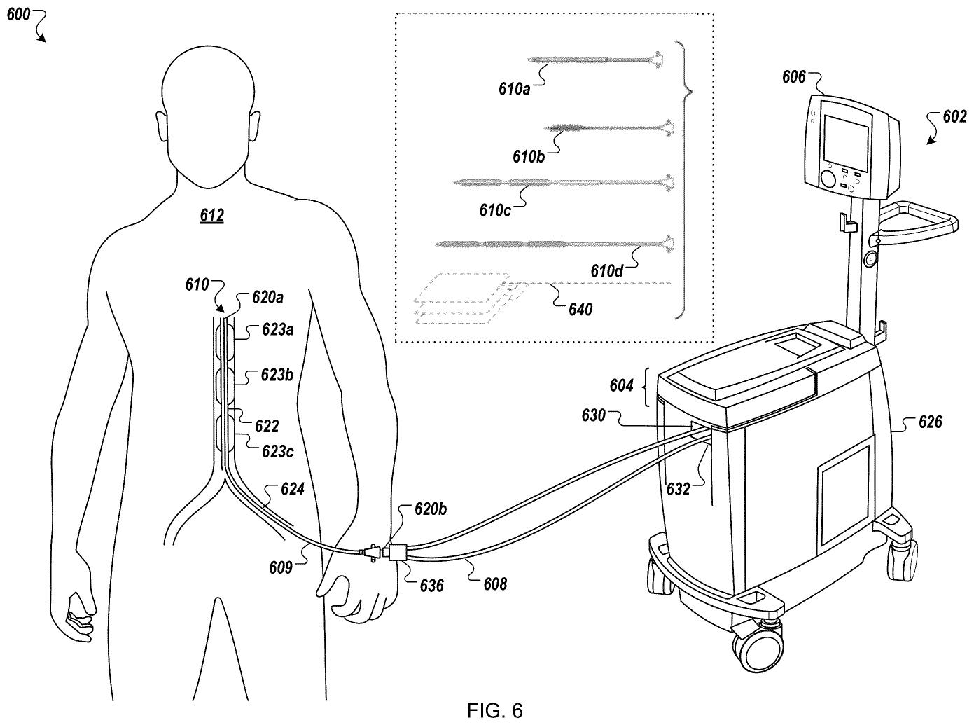

FIG. 6 is a diagram of an example temperature management system 600. The temperature management system 600 is configured to control a temperature of a patient's body 612 using a heat exchange device 610. The temperature management system 600 may be configured to heat and/or cool the patient 612 to manage the temperature of the patient 612. Managing the temperature of the patient 612 may be referred to as heat exchange treatment of the patient, temperature management therapy of the patient, heating/warming treatment of the patient, or cooling treatment of the patient.

The heat exchange device 610, in some embodiments, is an intravascular heat exchange catheter, e.g., 610a-610d, configured to be inserted into a vasculature of the patient 612. Heat exchanger catheters interoperable with the temperature management system 600, in some examples, can include a heat exchange catheter 610a commercially available as the Cool Line® Catheter (ZOLL Circulation, Inc., San Jose, Calif.), a heat exchange catheter 610b commercially available as the Solex 7® Catheter (ZOLL Circulation, Inc., San Jose, Calif.), a heat exchange catheter 610c commercially available as the Icy® Catheter (ZOLL Circulation, Inc., San Jose, Calif.), and/or a heat exchange catheter 610d commercially available as the Quattro® Catheter (ZOLL Circulation, Inc., San Jose, Calif).

In other embodiments, the heat exchange device 610 is a heat exchanger configured to be applied to the surface of the patient 612, such as a heat exchange pad (e.g., heat exchanger pad 640), a heat exchanger blanket, or a heat exchanger garment. The temperature management system 600 can include other hardware configured for heating and/or cooling the patient 612 such as, in some examples, heat exchange fluid loops, heating or cooling plates, heating or cooling cassettes, and/or heat exchange baths. The temperature management system 600, in some embodiments, is configured to measure patient data representing one or more physiological parameters of the patient, such as patient temperature, during treatment of the patient.

The temperature management system 600, in some implementations, includes an extracorporeal control console 602 with an interface 604 providing a coupling between the heat exchange device 610 and the control console 602 via a heat exchange fluid loop including tubing 608. The temperature management system 600 may be configured to control the temperature of the patient's body 612 based on operational data (e.g., pump speed, coolant temperature, and power) of the control console 602 and/or patient data (e.g., patient temperature feedback received from one or more temperature sensors located in or on the patient, such as temperature sensors 620a and/or 620b). The operational data and the patient data that are measured during treatment of the patient 612 may be referred to as treatment data.

In some implementations, the heat exchange device 610 couples to the control console 602 via a connector 636 connecting the tubing 608 to inflow and outflow tubes 609 of the heat exchange device 610. In some embodiments, the temperature management system 600 includes one or more single use elements, such as the tubing 608 of the fluid loop and/or the temperature sensors 620a, 620b.

As illustrated, the heat exchange device 610 includes an elongate catheter body 622 and a heat exchanger 623a-c positioned on a distal portion of the catheter body 622. The heat exchanger 623a-c, in some examples, may include an inflatable cylindrical balloon, a serpentine balloon or tubing, or a helical balloon or tubing through which thermal exchange fluid circulates. Inflow and outflow lumens (not illustrated) are present within the catheter body 622 to facilitate circulation of the thermal exchange fluid through the elongate catheter body 622. The catheter body 622 may include one or more working lumens 624 extending through the catheter body 622 and terminating distally at one or more openings in the distal end of the catheter body. The working lumens 624 may serve, for example, as a guidewire lumen to facilitate insertion and positioning of the catheter 622. In another example, the working lumens 624 may be used after insertion of the catheter 622 for delivery of fluids, medicaments or other devices. In illustration, the temperature sensor 620a may be inserted through the working lumen 624 and advance out of the distal end opening to a location beyond the distal end of the catheter body 622.

The extracorporeal control console 602 includes, generally, a main housing 626 and a console head including a user interface 606. The main housing 626 may contain various apparatuses and circuitry for warming/cooling thermal exchange fluid such as, in some examples, coolant, refrigerant, and/or saline. The main housing 626 may also include apparatuses and circuitry for pumping the thermal exchange fluid through the thermal exchange device 610 to effectively modify and/or control the patient's body temperature. The housing 626 may further include connection ports 630 and 632 for connection of additional or alternative types of temperature sensors and/or other apparatuses.

In some implementations, the user interface 606 is configured for accepting input data or control signals to the temperature management system 600 and for presenting information, such as treatment data indicative of treatment of the patient 612, to a clinician or other user. The user interface 606 may include a display screen, one or more physical controls, and/or virtual controls of a touch screen system. The temperature management system 600 may be configured to display, at the user interface 606, an operational status of the temperature management system 600 and a physiological status of the patient 612 during temperature management therapy. The operational status can include whether the temperature management system 600 is working at a maximum cooling or heating power (e.g., work or effort) or a percentage of the maximum heating or cooling power. The operational status can additionally include an alarm status, such as a low alarm limit (illustrated in FIG. 3A and FIG. 3F above) or a TrakLo alarm limit (illustrated in FIG. 3D and FIG. 3E above).

In some embodiments, the user interface 606 displays setup information, such as the settings displays illustrated in FIG. 3B, FIG. 3C, and/or FIG. 4, described above. The settings, for example, can include alarm limits, such as a high patient temperature alarm limit, a low patient temperature alarm limit, and/or a tracking patient temperature alarm limit (e.g., low or high).

One or more processors can be utilized to implement various functions and/or algorithms described herein. Additionally, any functions and/or algorithms described herein can be performed upon one or more virtual processors. The virtual processors, for example, may be part of one or more physical computing systems such as a computer farm or a cloud drive.

Aspects of the present disclosure may be implemented by software logic, including machine readable instructions or commands for execution via processing circuitry. The software logic may also be referred to, in some examples, as machine readable code, software code, or programming instructions. The software logic, in certain embodiments, may be coded in runtime-executable commands and/or compiled as a machine-executable program or file. The software logic may be programmed in and/or compiled into a variety of coding languages or formats.

Aspects of the present disclosure may be implemented by hardware logic (where hardware logic naturally also includes any necessary signal wiring, memory elements and such), with such hardware logic able to operate without active software involvement beyond initial system configuration and any subsequent system reconfigurations (e.g., for different object schema dimensions). The hardware logic may be synthesized on a reprogrammable computing chip such as a field programmable gate array (FPGA) or other reconfigurable logic device. In addition, the hardware logic may be hard coded onto a custom microchip, such as an application-specific integrated circuit (ASIC). In other embodiments, software, stored as instructions to a non-transitory computer-readable medium such as a memory device, on-chip integrated memory unit, or other non-transitory computer-readable storage, may be used to perform at least portions of the herein described functionality.

Various aspects of the embodiments disclosed herein are performed on one or more computing devices, such as a laptop computer, tablet computer, mobile phone or other handheld computing device, or one or more servers. Such computing devices include processing circuitry embodied in one or more processors or logic chips, such as a central processing unit (CPU), graphics processing unit (GPU), field programmable gate array (FPGA), application-specific integrated circuit (ASIC), or programmable logic device (PLD). Further, the processing circuitry may be implemented as multiple processors cooperatively working in concert (e.g., in parallel) to perform the instructions of the inventive processes described above.

The process data and instructions used to perform various methods and algorithms derived herein may be stored in non-transitory (i.e., non-volatile) computer-readable medium or memory. The claimed advancements are not limited by the form of the computer-readable media on which the instructions of the inventive processes are stored. For example, the instructions may be stored on CDs, DVDs, in FLASH memory, RAM, ROM, PROM, EPROM, EEPROM, hard disk or any other information processing device with which the computing device communicates, such as a server or computer. The processing circuitry and stored instructions may enable the computing device to perform, in some examples, the method 200 of FIG. 2A-1 and FIG. 2A-2, the method 230 of FIG. 2B-1 and FIG. 2B-2, and/or the method 260 of FIG. 2C.

These computer program instructions can direct a computing device or other programmable data processing apparatus to function in a particular manner, such that the instructions stored in the computer-readable medium produce an article of manufacture including instruction means which implement the function/operation specified in the illustrated process flows.

Embodiments of the present description rely on network communications. As can be appreciated, the network can be a public network, such as the Internet, or a private network such as a local area network (LAN) or wide area network (WAN) network, or any combination thereof and can also include PSTN or ISDN sub-networks. The network can also be wired, such as an Ethernet network, and/or can be wireless such as a cellular network including EDGE, 3G, 4G, and 5G wireless cellular systems. The wireless network can also include Wi-Fi®, Bluetooth®, Zigbee®, or another wireless form of communication. The network, for example, may support communications between one or more of the patient sensors 512 and the temperature management control unit 502, the temperature management control unit 502, one or more of the and/or environmental sensors 514 and the temperature management control unit 502, and/or the temperature management control unit 502 and the computing system 530 of FIG. 5.

The computing device, in some embodiments, further includes a display controller for interfacing with a display, such as a built-in display or LCD monitor. A general purpose I/O interface of the computing device may interface with a keyboard, a hand-manipulated movement tracked I/O device (e.g., mouse, virtual reality glove, trackball, joystick, etc.), and/or touch screen panel or touch pad on or separate from the display. The display controller and display may enable presentation of the user interfaces illustrated, in some examples, in the graph 100 of FIG. 1A, the graph 110 of FIG. 1B, the graph 120 of FIG. 1C, the graph 130 of FIG. 1D, the graphic display 300 of FIG. 3A, the graphic display 320 of FIG. 3B, the graphic display 330 of FIG. 3C, the graphic display 340 of FIG. 3D, the graphic display 350 of FIG. 3E, the graphic display 360 of FIG. 3F, and/or the graphic displays 400 of FIG. 4.

Moreover, the present disclosure is not limited to the specific circuit elements described herein, nor is the present disclosure limited to the specific sizing and classification of these elements. For example, the skilled artisan will appreciate that the circuitry described herein may be adapted based on changes in battery sizing and chemistry or based on the requirements of the intended back-up load to be powered.

The functions and features described herein may also be executed by various distributed components of a system. For example, one or more processors may execute these system functions, where the processors are distributed across multiple components communicating in a network. The distributed components may include one or more client and server machines, which may share processing, in addition to various human interface and communication devices (e.g., display monitors, smart phones, tablets, personal digital assistants (PDAs)). The network may be a private network, such as a LAN or WAN, or may be a public network, such as the Internet. Input to the system, in some examples, may be received via direct user input and/or received remotely either in real-time or as a batch process.

Although provided for context, in other implementations, methods and logic flows described herein may be performed on modules or hardware not identical to those described. Accordingly, other implementations are within the scope that may be claimed.

In some implementations, a cloud computing environment, such as Google Cloud Platform™ or Amazon™ Web Services (AWS™), may be used perform at least portions of methods or algorithms detailed above. The processes associated with the methods described herein can be executed on a computation processor of a data center. The data center, for example, can also include an application processor that can be used as the interface with the systems described herein to receive data and output corresponding information. The cloud computing environment may also include one or more databases or other data storage, such as cloud storage and a query database. In some implementations, the cloud storage database, such as the Google™ Cloud Storage or Amazon™ Elastic File System (EFS™), may store processed and unprocessed data supplied by systems described herein. For example, the contents of storage regions of the computing system 530 of FIG. 5 may be maintained in a database structure.

The systems described herein may communicate with the cloud computing environment through a secure gateway. In some implementations, the secure gateway includes a database querying interface, such as the Google BigQuery™ platform or Amazon RDS™. The data querying interface, for example, may support access by the temperature management control unit 502 of FIG. 5.

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1. A patient temperature management system for monitoring patient temperature during delivery of temperature management therapy, the system comprising:
a non-volatile computer-readable memory storing a target patient temperature; and
processing circuitry configured to perform operations comprising
activating a patient temperature tracking mode, wherein, while in the patient temperature tracking mode, a temperature threshold alert corresponding to a temperature tracking threshold is enabled,
initializing the temperature tracking threshold based on an initial patient temperature measured using temperature data from at least one temperature sensor, wherein the temperature tracking threshold is set to be within a predetermined value of the initial patient temperature, and
repeatedly, during temperature management therapy configured to adjust the initial patient temperature to a target patient temperature,
obtaining the current patient temperature based on temperature data from the at least one temperature sensor, and
based at least in part on determining the current patient temperature is indicative of movement toward the target patient temperature, updating the temperature tracking threshold to be within the predetermined value of the current patient temperature.

2. The system of claim 1, further comprising a temperature management device configured to perform the temperature management therapy.

3. The system of claim 1, further comprising at least one output device for generating an alarm for an operator of the patient temperature management system corresponding to the temperature tracking threshold.

4. The system of claim 3, wherein the operations further comprise, based at least in part on the current patient temperature being further away from the target patient temperature than a current value of the temperature tracking threshold, issuing an audible alert via the at least one output device.

5. The system of claim 1, further comprising the at least one temperature sensor.

6. The system of claim 1, further comprising a display device, wherein
the patient temperature tracking mode is activated based on a user input setting at an interactive user interface presented on the display device.

7. The system of claim 6, wherein the operations further comprise presenting, at the display device, a current value of the temperature tracking threshold.

8. The system of claim 7, wherein presenting the current value of the temperature tracking threshold comprises presenting the current value on a line graph presenting historic values of the temperature tracking threshold.

9. The system of claim 6, wherein the operations further comprise, based at least in part on the current patient temperature being further away from the target patient temperature than a current value of the temperature tracking threshold, presenting an alert on the display device.

10. The system of claim 1, wherein activating the patient temperature tracking mode comprises automatically activating the patient temperature tracking mode based at least in part on the initial patient temperature being at least a threshold value further away from the target patient temperature than a patient temperature limit.

11. The system of claim 1, wherein activating the patient temperature tracking mode comprises receiving user selection of a setting corresponding to the patient temperature tracking mode.

12. (canceled)

13. The system of claim 1, wherein:
while in the patient temperature tracking mode, a temperature limit alert corresponding to a patient temperature limit is disabled; and
when the current patient temperature meets or moves beyond the patient temperature limit, the temperature limit alert is automatically enabled and the temperature tracking threshold is automatically disabled.

14. The system of claim 13, wherein the temperature limit alert is automatically enabled when the current patient temperature moves beyond the patient temperature limit by at least the predetermined value.

15. A method for monitoring patient temperature during delivery of temperature management therapy, the method comprising:
activating, by processing circuitry, a patient temperature tracking mode, wherein, while in the patient temperature tracking mode, a temperature threshold alert corresponding to a temperature tracking threshold is enabled;
initializing, by the processing circuitry, the temperature tracking threshold based on an initial patient temperature measured using temperature data from at least one temperature sensor, wherein
the temperature tracking threshold is set to be within a predetermined value of the initial patient temperature; and
repeatedly, during temperature management therapy configured to adjust the initial patient temperature to a target patient temperature,
obtaining, by the processing circuitry, a current patient temperature based on temperature data from the at least one temperature sensor, and
analyzing, by the processing circuitry, the current patient temperature at least in view of the target patient temperature, wherein the analyzing comprises, based at least in part on determining the current patient temperature is indicative of movement toward closer to the target patient temperature, updating the temperature tracking threshold to be within the predetermined value of the current patient temperature.

16. The method of claim 15, wherein analyzing the current patient temperature at least in view of the target patient temperature comprises, based at least in part on the current patient temperature meeting or moving beyond a patient temperature limit,
automatically enabling a temperature limit alert, and
automatically disabling the temperature tracking threshold.

17. The method of claim 15, wherein a temperature management therapy apparatus comprises the processing circuitry.

18.-19. (canceled)

20. The method of claim 15, wherein, while in the patient temperature tracking mode, a temperature limit alert corresponding to a patient temperature limit is disabled.

21. The method of claim 15, wherein the analyzing comprises, based at least in part on the current patient temperature being further away from the target patient temperature than a current value of the temperature tracking threshold, issuing an alert for a caregiver or operator of the temperature management therapy.

22.-27. (canceled)

28. The system of claim 2, wherein the temperature management device comprises an endovascular catheter.

29. The system of claim 2, wherein the temperature management device comprises a surface heat exchange device.

30.-58. (canceled)
